SEQUENCE LISTING (5 sequences)

SEQ ID NO: 1 - DNA, 15 bp, synthetic construct
  source 1..15: mol_type = other DNA
catttcccgtaaatc

SEQ ID NO: 2 - DNA, 15 bp, synthetic construct
  source 1..15: mol_type = other DNA
gatttacgggaaatg

SEQ ID NO: 3 - DNA, 115 bp, synthetic construct
  source 1..115: mol_type = other DNA
  variation 1..50: note = Wherein up to 50 copies of n may be omitted
  variation 66..115: note = Wherein up to 50 copies of n may be omitted
  variation 53: note = Wherein the n at position 53 may be thymine
  variation 57: note = Wherein the n at position 57 may be cytosine
  variation 59: note = Wherein the n at position 59 may be guanine
  variation 61: note = Wherein the n at position 61 may be adenine
  variation 63: note = Wherein the n at position 61 may be adenine
nnnnnnnnnnnnnnnnnnnnnnnnnnnnnnnnnnnnnnnnnnnnnnnnnncanttcncntnantcnnnnnnnnnnnnnnnnnnnnnnnnnnnnnnnnnnnnnnnnnnnnnnnnnn

SEQ ID NO: 4 - DNA, 15 bp, synthetic construct
  source 1..15: mol_type = other DNA
cttttcccgtaaatc

SEQ ID NO: 5 - DNA, 15 bp, synthetic construct
  source 1..15: mol_type = other DNA
gatttacgggaaaag

(19) United States
(12) Patent Application Publication
(10) Pub. No.: US 2026/0085318 A1
(43) Pub. Date: Mar. 26, 2026

(54) Methods for Treating Cancer Using a Stat3 Double-Stranded, Cyclic Oligonucleotide Decoy

(71) Applicant: The Regents of the University of California, Oakland, CA (US)

(72) Inventors: Daniel E. Johnson, San Francisco, CA (US); Jennifer R. Grandis, San Francisco, CA (US)

(21) Appl. No.: 19/112,832

(22) PCT Filed: Sep. 20, 2023
(86) PCT No.: PCT/US2023/074705

§ 371 Date: Mar. 18, 2025

Related U.S. Application Data
(60) Provisional application No. 63/409,520, filed on Sep. 23, 2022.

(51) Int. Cl. C12N 15/113; A61K 31/713; A61K 39/00; A61P 35/00; C07K 16/28

(52) U.S. Cl. CPC C12N 15/1136; A61K 31/713; A61P 35/00; C07K 16/2818; C07K 16/2827; A61K 2039/505; C07K 2317/76; C12N 2310/11

(57) ABSTRACT

The present invention provides methods of treating cancer using a STAT3 double-stranded, cyclic oligonucleotide decoy in combination with an immune checkpoint inhibitor.

### CROSS-REFERENCE

This application claims the benefit of U.S. Provisional Application No. 63/409,520 filed Sep. 23, 2022, which application is incorporated herein by reference in its entirety.

### REFERENCE TO AN ELECTRONIC SEQUENCE LISTING

The contents of the electronic sequence listing (UCSF-685WO_SEQ_LIST.xml; Size: 8,437 bytes; and Date of Creation: Sep. 20, 2023) are herein incorporated by reference in its entirety.

### FIELD OF THE INVENTION

The present invention provides methods of treating cancer using a STAT3 double-stranded, cyclic oligonucleotide decoy in combination with an immune checkpoint inhibitor.

### BACKGROUND

Cancer continues to be a significant health problem despite the substantial research efforts and scientific advances reported in the literature for treating this disease. Solid tumors, such as prostate cancer, colorectal cancer, skin cancer, head and neck cancer, breast cancer, and lung cancer remain highly prevalent among the world population. Existing therapies for treating cancer include localized therapies, such as surgery, radiation therapy, cryotherapy, and systemic therapies (e.g., chemotherapy, hormonal therapy, immune therapy, targeted therapy, and cell therapy) used alone or in combination. Support therapies are also used in some contexts, where supportive therapies are additional treatments that do not directly treat cancer but are used to reduce side effects and address patient quality of life. However, current treatment options for cancer are not effective for all patients and/or can have substantial adverse side effects. New therapies are needed to address this unmet need in cancer therapy.

Signal transducer and activator of transcription 3 (STAT3) is a transcription factor that is broadly hyperactivated in cancer and non-cancerous cells within the tumor microenvironment. STAT3 activation has two distinct roles in tumor cells and in the immune cells of the tumor microenvironment. Activated STAT3 increases the levels of transcription of several oncogenes such as Cyclin D1 and BcL-XL. STAT3 also has an important role in inhibiting the expression of various immune activation regulators and promoting production of immunosuppressive factors. For example, in tumor cells, hyperactivated STAT3 decreases the expression of immune-stimulating factors including interferons, pro-inflammatory cytokines (e.g., IL-12 and TNF-α) and chemokines (e.g., CCL5 and CXCL10). STAT3 can also interact with other signaling pathways, such as NF-κB, to result in tumor progression. Activation of STAT3 in tumor infiltrating immune cells results in potent immunosuppressive effects, including negative regulation of neutrophil and natural killer (NK) cell function, induction of PD-1 and inhibition of effector T cell function, inhibition of dendritic cell (DC) maturation and function, and expansion of T regulatory (Treg) and myeloid-derived suppressor cells (MDSCs). Oligonucleotide molecules that inhibit STAT3 are described in, for example, U.S. Pat. No. 8,722,640. Improved methods for using STAT3 inhibitors to treat cancer would benefit patients.

Accordingly, a need exists for improved treatments for cancer. The present invention addresses this need and provides other related advantages.

### SUMMARY OF THE INVENTION

The present invention provides methods of treating cancer using a STAT3 double-stranded, cyclic oligonucleotide decoy in combination with an immune checkpoint inhibitor. In one aspect, the invention provides a method of treating cancer in a patient. The method comprises administering to a patient in need thereof a therapeutically effective amount of a STAT3 double-stranded, cyclic oligonucleotide decoy and an immune checkpoint inhibitor, to treat the cancer. The immune checkpoint inhibitor may be, for example, a PD-1 inhibitor.

### DETAILED DESCRIPTION OF THE INVENTION

The present invention provides methods of treating cancer using a STAT3 double-stranded, cyclic oligonucleotide decoy in combination with an immune checkpoint inhibitor. The method may be further characterized according to identity of the STAT3 double-stranded, cyclic oligonucleotide decoy, the immune checkpoint inhibitor, achievements in efficacy in treating the cancer, and other features. Various aspects of the invention are set forth below in sections; however, aspects of the invention described in one particular section are not to be limited to any particular section.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”. 5^{th} Ed., Ed.: Smith, M. B. and March, J., John Wiley & Sons, New York: 2001, the entire contents of which are hereby incorporated by reference.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

The term “double-stranded” refers to a nucleic acid molecule comprising two complementary nucleotide strands connected to each other by Watson-Crick base pairing.

As used herein, the terms “five prime” or “5′” and “three-prime” or “3′” refer to a specific orientation as related to a nucleic acid. Nucleic acids have a distinct chemical orientation such that their two ends are distinguished as either five-prime (5′) or three-prime (3′). The 3′ end of a nucleic acid contains a free hydroxyl group attached to the 3′ carbon of the terminal pentose sugar. The 5′ end of a nucleic acid contains a free hydroxyl or phosphate group attached to the 5′ carbon of the terminal pentose sugar. In the case of double stranded DNA, each of the strands has a 5′ and a 3′-end and the strands are in opposite orientation to each other.

The terms “a,” “an” and “the” as used herein mean “one or more” and include the plural unless the context is inappropriate.

The term “about” means within 10% of the stated value. In certain embodiments, the value may be within 8%, 6%, 4%, 2%, or 1% of the stated value.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

The term “ethylene glycolyl” refers to the chemical fragment —OCH₂CH₂—.

The term “hexaethylene glycolyl” refers to the chemical fragment —(OCH₂CH₂)₆—.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Therapeutic Methods

The present invention provides methods of treating cancer using a STAT3 double-stranded, cyclic oligonucleotide decoy in combination with an immune checkpoint inhibitor. Aspects of the therapeutic methods are described in more detail below, along with description of STAT3 double-stranded, cyclic oligonucleotide decoys for use in the therapeutic methods.

One aspect of the invention provides a method of treating cancer in a patient. The method comprises administering to a patient in need thereof a therapeutically effective amount of a STAT3 double-stranded, cyclic oligonucleotide decoy and an immune checkpoint inhibitor, to treat the cancer.

Additional exemplary features that may be used to further characterize the method are described herein including, for example, the identity of the STAT3 double-stranded, cyclic oligonucleotide decoy, the immune checkpoint inhibitor, identity of the cancer, and other features. A more thorough description of these and other features is provided below.

### STAT3 Double-Stranded Cyclic Oligonucleotide Decoy

The methods may be further characterized according to the identity of the STAT3 double-stranded, cyclic oligonucleotide decoy. For example, in certain embodiments, the STAT3 double-stranded, cyclic oligonucleotide decoy may be further characterized according to the length of the sense strand. For example, in certain embodiments, the sense strand is up to 18 nucleotides long. In certain embodiments, the sense strand is up to 17 nucleotides long. In certain embodiments, the sense strand is up to 16 nucleotides long. In certain embodiments, the sense strand is up to 15 nucleotides long.

The STAT3 double-stranded, cyclic oligonucleotide decoy may be further characterized according to the nucleotide sequence of the sense strand. For example, in certain embodiments, the sense strand consists of the sequence 5′-CATTTCCCGTAAATC-3′ (SEQ ID NO: 1).

The STAT3 double-stranded, cyclic oligonucleotide decoy may be further characterized according to the identity of the antisense strand. For example, in certain embodiments, the antisense strand is at least partially complementary to the sense strand. In certain embodiments, the antisense strand is fully complementary to the sense strand. In certain embodiments, the antisense strand is at least partially complementary to the sense strand by Watson-Crick base pairing. In certain embodiments, the antisense strand is fully complementary to the sense strand by Watson-Crick base pairing. In certain embodiments, antisense strand comprises the sequence 3′-GTAAAGGGCATTTAG-5′ (SEQ ID NO: 2). In certain embodiments, antisense strand consists of the sequence 3′-GTAAAGGGCATTTAG-5′ (SEQ ID NO: 2).

The STAT3 double-stranded, cyclic oligonucleotide decoy may be further characterized according to nature of the backbone of the sense strand and/or antisense strand. For example, in certain embodiments, the sense strand has a phosphodiester backbone. In certain embodiments, the antisense strand has a phosphodiester backbone.

The STAT3 double-stranded, cyclic oligonucleotide decoy may be further characterized according to the identity of the first carbon-containing linker and/or the second carbon-containing linker. For example, in certain embodiments, the first carbon-containing linker comprises ethylene glycolyl. In certain embodiments, the first carbon-containing linker comprises one or more ethylene glycolyl. In certain embodiments, the first carbon-containing linker comprises hexaethylene glycolyl. In certain embodiments, the first carbon-containing linker is hexaethylene glycolyl. In certain embodiments, the second carbon-containing linker comprises ethylene glycolyl. In certain embodiments, the second carbon-containing linker comprises one or more ethylene glycolyl. In certain embodiments, the second carbon-containing linker comprises hexaethylene glycolyl. In certain embodiments, the second carbon-containing linker is hexaethylene glycolyl.

In certain embodiments, the STAT3 double-stranded, cyclic oligonucleotide decoy has a serum half-life of greater than about 1.5 hours, 2 hours, 3 hours, 4 hours, 5 hours, 6 hours, 7 hours, 8 hours, 12 hours, 20 hours, 24 hours, or 48 hours.

In certain embodiments, the STAT3 double-stranded, cyclic oligonucleotide decoy is compound 1, which is a STAT3 double-stranded oligonucleotide decoy having a sense strand, an antisense strand, a first linker comprising hexaethylene glycolyl wherein the first linker binds the 3′ end of the sense strand to the 5′ end of the antisense strand, and a second linker comprising hexaethylene glycolyl wherein the second linker binds the 5′ end of the sense strand to the 3′ end of the antisense strand, wherein the sense strand has the sequence 5′-CATTTCCCGTAAATC-3′ (SEQ ID NO: 1), and the antisense strand has the sequence 3′-GTAAAGGGCATTTAG-5′ (SEQ ID NO: 2). See, for example, U.S. Pat. No. 8,722,640. which is hereby incorporated by reference.

### Additional STAT3 Double-Stranded, Cyclic Oligonucleotide Decoys

Additional STAT3 double-stranded, cyclic oligonucleotide decoys useful for methods described herein include:
(a) a cyclic double-stranded STAT3 oligonucleotide decoy, wherein (i) the decoy comprises an oligonucleotide, or an analog thereof, having the sequence 5′-(N₆)ₙCAN₁TTCN₂CN₃TN₄AN₅T C-(N₇)ₘ-3′ (SEQ ID NO: 3), wherein N₁, N₂, N₃, N₄ and N₅ are A, T, G or C; and one, two, three or all of the following conditions are met: N₁, is T, N₂ is C, N₃ is G, N₄ is A, N₅ is A, and N₆ and N₇ are A, T, G or C, and n and m are independently 0-50, and (ii) the two strands are joined by spacers at both ends; and
(b) a cyclic double-stranded STAT3 oligonucleotide decoy, wherein (i) the decoy comprises an oligonucleotide, or an analog thereof, having the sequence 5′-(N₆)ₙCAN₁TTCN₂CN₃TN₄AN₅TC-(N₇)ₘ-3′ (SEQ ID NO: 3), wherein N₃ is G; N₁, N₂, and N₄ are A, T, G or C; one, two, three or all of the following conditions are met: N₁, is T, N₂ is C, N₄ is A, and Nₛ is A, and N₆ and N₇ are A, T, G or C, and n and m are independently 0-50, (ii) the two strands are joined by hexaethylene glycolyl spacers at both ends; (iii) the decoy binds to STAT3 protein under physiologic conditions and interferes with STAT3 binding to its target sequence; and (iv) the decoy has a serum half-life of greater than about 4 hours.

The cyclic double-stranded STAT3 oligonucleotide decoy in Part (a) above may be characterized by additional features. For example, in certain embodiments, the decoy binds to STAT3 protein under physiologic conditions and interferes with STAT3 binding to its target sequence. In certain embodiments, the decoy has a serum half-life of greater than about 4 hours. In certain embodiments, the spacers are hexaethylene glycolyl spacers. In certain embodiments, said STAT3 protein is dimerized STAT3 protein.

The cyclic double-stranded STAT3 oligonucleotide decoy in Part (a) and Part (b) above may be characterized by additional features. For example, in certain embodiments, the oligonucleotide comprises sequence 5′-CATTTCCCGTAAATC-3′ (SEQ ID NO: 1). In certain embodiments, the oligonucleotide comprises a 15-mer oligonucleotide sequence, wherein the sequence is 5′-CATTTCCCGTAAATC-3′ (SEQ ID NO: 1).

In certain embodiments, N₂ is a pyrimidine. In certain embodiments, at least two of the following are met: N₁, is T; N₂ is C; N₄ is A and N₅ is A. In certain embodiments, at least three of the following are met: N₁ is T; N₂ is C; N₄ is A and N₅ is A.

In certain embodiments, the cyclic double-stranded STAT3 oligonucleotide decoy has a serum half-life of greater than about 1.5 hours, 2 hours, 3 hours, 4 hours, 5 hours, 6 hours, 7 hours, 8 hours, 12 hours, 20 hours, 24 hours, or 48 hours.

Further description of STAT3 double-stranded, cyclic oligonucleotide decoys are described in, for example, U.S. Pat. No. 8,722,640, which is hereby incorporated by reference.

### Immune Checkpoint Inhibitor

The methods may be further characterized according to the identity of immune checkpoint inhibitor. For example, in certain embodiments, the immune checkpoint inhibitor is an inhibitor of PD-1. In certain embodiments, the immune checkpoint inhibitor is an anti-PD-1 antibody. In certain embodiments, the immune checkpoint inhibitor is an anti-PD-1 antibody selected from pembrolizumab, nivolumab, cemiplimab, atezolizumab, dostarlimab, durvalumab, or avelumab. In certain embodiments, the immune checkpoint inhibitor is pembrolizumab. In certain embodiments, the immune checkpoint inhibitor is nivolumab. In certain embodiments, the immune checkpoint inhibitor is cemiplimab. In certain embodiments, the immune checkpoint inhibitor is atezolizumab. In certain embodiments, the immune checkpoint inhibitor is dostarlimab. In certain embodiments, the immune checkpoint inhibitor is durvalumab. In certain embodiments, the immune checkpoint inhibitor is avelumab.

In certain embodiments, the immune checkpoint inhibitor is an inhibitor of PD-L1. In certain embodiments, the immune checkpoint inhibitor is an anti-PD-L1 antibody. In certain embodiments, the immune checkpoint inhibitor is an anti-PD-L1 antibody selected from atezolizumab, avelumab, or durvalumab. In certain embodiments, the immune checkpoint inhibitor is atezolizumab. In certain embodiments, the immune checkpoint inhibitor is avelumab. In certain embodiments, the immune checkpoint inhibitor is durvalumab.

In certain embodiments, the immune checkpoint inhibitor is an anti-PD-1 antibody or an anti-PD-L1 antibody that inhibits binding of PD-1 to PD-L1.

### Type of the Cancer

The methods may be further characterized according to the type of the cancer. For example, in certain embodiments, the cancer is a solid tumor. In certain embodiments, the cancer is a head and neck cancer, breast cancer, ovarian cancer, lung cancer, pancreatic cancer, prostate cancer, skin cancer, cervical cancer, oral cancer, esophageal cancer, bladder cancer, leukemia, lymphoma, or a glioma. In certain embodiments, the cancer is multiple myeloma, HTLV-1 dependent leukemia, acute myelogenous leukemia, large granular lymphocyte leukemia, lymphoma, EBV-related Burkitt's lymphoma, mycosis fungoides, cutaneous T-cell lymphoma, non-Hodgkins lymphoma, anaplastic large-cell lymphoma, breast cancer, melanoma, ovarian cancer, lung cancer, pancreatic cancer, or prostate cancer.

In certain embodiments, the cancer is a cancer of the cervix; penis; head and neck, including, without limitation cancers of the oral cavity, salivary glands, paranasal sinuses and nasal cavity, pharynx and larynx; lung; esophageal; skin; or vulva or bladder.

In certain embodiments, the cancer is a head and neck cancer. In certain embodiments, the cancer is a cancer located in the head or neck. In certain embodiments, the cancer is lung cancer. In certain embodiments, the cancer is non-small cell lung cancer. In certain embodiments, the cancer is colorectal cancer, pancreatic cancer, ovarian cancer, or melanoma. In certain embodiments, the cancer is colorectal cancer. In certain embodiments, the cancer is pancreatic cancer. In certain embodiments, the cancer is ovarian cancer. In certain embodiments, the cancer is melanoma.

In certain embodiments, the cancer is a squamous cell carcinoma. In certain embodiments, the cancer is a head and neck squamous cell carcinoma (HNSCC).

In certain embodiments, the cancer is recurrent. In certain embodiments, the cancer is metastatic.

In certain embodiments, the cancer is an epithelial cancer.

### Aspects of Administration

The methods may be further characterized according to aspects of the administration. For example, in certain embodiments, the STAT3 double-stranded, cyclic oligonucleotide decoy is administered simultaneously with the immune checkpoint inhibitor. In certain embodiments, the STAT3 double-stranded, cyclic oligonucleotide decoy and immune checkpoint inhibitor are administered sequentially.

In certain embodiments, the STAT3 double-stranded, cyclic oligonucleotide decoy and/or an immune checkpoint inhibitor is administered intravenously to the patient.

Other routes of administration, systemic or local, may also be used. For example, the routes for administration of a STAT3 double-stranded, cyclic oligonucleotide decoy and an immune checkpoint inhibitor (e.g., an anti-PD-1 antibody) may be independently selected from intravenous, intraarterial, intramuscular, intranasal, intratracheal, subcutaneous, or intradermal. In certain embodiments, a composition comprising a STAT3 double-stranded, cyclic oligonucleotide decoy and/or an immune checkpoint inhibitor may be injected directly into a tumor.

Patient Populations that May Derive Particular Benefits from the Therapeutic Methods

The methods may be further characterized according to patient populations that may derive particular benefits from the therapeutic methods. For example, in certain embodiments, the patient is a human. In certain embodiments, the patient is an adult human. In certain embodiments, the patient is a geriatric human. In certain embodiments, the patient is a pediatric human.

### Characterization of Result Achieved

The methods may be further characterized according to result achieved. For example, in certain embodiments, there is at least a 20% further reduction in tumor size compared to the result achieved using the STAT3 double-stranded, cyclic oligonucleotide decoy as monotherapy at the same dose amount. In certain embodiments, there is at least a 30%, 40%, 50%, 60%, 70%, 80% or 90% further reduction in tumor size compared to the result achieved using the STAT3 double-stranded, cyclic oligonucleotide decoy as monotherapy at the same dose amount. In certain embodiments, the further reduction in tumor size is measured at a point in time that is 1, 2, 3, 4, 5, 6, 7, 8, 9, 10, 11, or 12 months from the start of therapy using the STAT3 double-stranded, cyclic oligonucleotide decoy.

In certain embodiments, there is at least a 20% further reduction in tumor size compared to the result achieved using the immune checkpoint inhibitor as monotherapy at the same dose amount. In certain embodiments, there is at least a 30%, 40%, 50%, 60%, 70%, 80% or 90% further reduction in tumor size compared to the result achieved using the immune checkpoint inhibitor as monotherapy at the same dose amount. In certain embodiments, the further reduction in tumor size is measured at a point in time that is 1, 2, 3, 4, 5, 6, 7, 8, 9, 10, 11, or 12 months from the start of therapy using the STAT3 double-stranded, cyclic oligonucleotide decoy.

In certain embodiments, there is at least a 20% improvement in the average duration of patient survival compared to the result achieved using the STAT3 double-stranded, cyclic oligonucleotide decoy as monotherapy at the same dose amount. In certain embodiments, there is at least a 30%, 40%, 50%, 60%, 70%, 80%, 90%, 100%, 150%, or 200% improvement in the average duration of patient survival compared to the result achieved using the STAT3 double-stranded, cyclic oligonucleotide decoy as monotherapy at the same dose amount.

In certain embodiments, there is at least a 20% improvement in the average duration of patient survival compared to the result achieved using the immune checkpoint inhibitor as monotherapy at the same dose amount. In certain embodiments, there is at least a 30%, 40%, 50%, 60%, 70%, 80%, 90%, 100%, 150%, or 200% improvement in the average duration of patient survival compared to the result achieved using the immune checkpoint inhibitor as monotherapy at the same dose amount.

### II. Pharmaceutical Compositions

Compounds described herein, such as a STAT3 double-stranded, cyclic oligonucleotide decoy and/or an immune checkpoint inhibitor, may be formulated as a pharmaceutical composition. Such pharmaceutical composition may provide benefits to patients. Pharmaceutical compositions may comprise a compound described herein, such as a STAT3 double-stranded, cyclic oligonucleotide decoy, and a pharmaceutically acceptable carrier. The pharmaceutical compositions may be specially formulated for administration in liquid form, including those adapted for intravenous administration.

The pharmaceutical compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration, and other factors. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, the formulation comprises a lipid nanoparticle.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Pharmaceutical compositions of this invention suitable for injection into the patient comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, saline, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsuled matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes, exosome, or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

### III. Compositions for Medical Use

Another aspect of the invention provides for the use of a compound described herein (such as a STAT3 double-stranded, cyclic oligonucleotide decoy or other compound in Section I) for treating a medical disorder, such a medical disorder described herein (e.g., cancer).

### IV. Preparation of a Medicament

Another aspect of the invention provides for the use of a compound described herein (such as a STAT3 double-stranded, cyclic oligonucleotide decoy or other compound in Section I) in the manufacture of a medicament. In certain embodiments, the medicament is for treating a disorder described herein, such as cancer.

### V. Medical Kits

Another aspect of the invention provides a medical kit comprising, for example, (i) a composition or dosage form comprising a compound described herein, and (ii) instructions for treating cancer according to methods described herein.

### EXAMPLES

### Example 1: Treatment of HNSCC Tumor with Cyclic STAT3 Decoy (CS3D) and Anti-PD-1 Antibody

Immunocompetent mice harboring syngeneic murine head and neck squamous cell carcinoma (HNSCC) tumors were treated with an anti-PD-1 (anti-PD-1, clone RMP1-14 from BioXCell; catalogue number BE0146) in combination with either cyclic STAT3 decoy (CS3D) or mutant control CS3D (mCS3D). This anti-PD-1 antibody has been demonstrated to block the binding of PD-1 to its ligands in vivo. The findings are:
1. anti-PD-1+CS3D results in statistically significant decrease in tumor volume relative to anti-PD-1+mCS3D.
2. anti-PD-1+CS3D results in statistically significant decrease in immunosuppressive T regulatory cells relative to anti-PD-1+mCS3D.
3. anti-PD-1+CS3D results in statistically significant increase in interferon-gamma (IFNγ) levels (an indicator effector T cell activity) relative to anti-PD-1+mCS3D.

Sequence of the CS3D molecule is as follows (top strand, SEQ ID NO:1; bottom strand, SEQ ID NO:2):

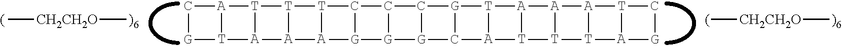

Sequence of the mCS3D molecule is as follows (top strand, SEQ ID NO:4; bottom strand, SEQ ID NO:5):

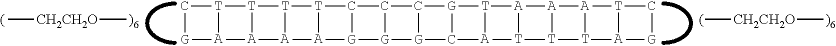

C57BL/6 mice were inoculated subcutaneously in the flank with 2-3 million syngeneic, murine MOC1 cells. When tumor volumes reached an average of 75 mm³ (day 1) mice were randomized into 4 treatment groups (n=5 mice/group). The following day, treatments began. Group one was treated with the murine version of mutant control CS3D plus isotype control antibody; group two was treated with the murine version of mutant control CS3D plus anti-PD-1; group three was treated with the murine version of CS3D plus isotype control antibody; group four was treated with the murine version of CS3D plus anti-PD-1. Mutant control CS3D and CS3D were administered 5 times/week at a dose of 5 mg/kg, via tail vein administration. Isotype control IgG and anti-PD-1 were administered at 250 micrograms/dose on days 2, 5, 8, and 12, via intraperitoneal administration. Tumor volumes were determined twice/week. Mice were euthanized on day 19 (Aug. 29, 2018), and tumors were harvested and processed for analysis. Flow cytometry was used to assess the levels of Tregulatory cells in the tumors, and ELISA kits were used to assess interferon gamma levels.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating cancer in a patient, comprising administering to a patient in need thereof a therapeutically effective amount of a STAT3 double-stranded, cyclic oligonucleotide decoy and an immune checkpoint inhibitor, to treat the cancer.

2. The method of claim 1, wherein the immune checkpoint inhibitor is an inhibitor of PD-1.

3. The method of claim 1, wherein the immune checkpoint inhibitor is an anti-PD-1 antibody.

4. The method of claim 1, wherein the immune checkpoint inhibitor is an anti-PD-1 antibody selected from pembrolizumab, nivolumab, cemiplimab, atezolizumab, dostarlimab, durvalumab, or avelumab.

5. The method of claim 1, wherein the immune checkpoint inhibitor is an inhibitor of PD-L1.

6. The method of claim 1, wherein the immune checkpoint inhibitor is an anti-PD-L1 antibody.

7. The method of claim 1, wherein the immune checkpoint inhibitor is an anti-PD-L1 antibody selected from atezolizumab, avelumab, or durvalumab.

8. The method of any one of claims 1-7, wherein the STAT3 double-stranded, cyclic oligonucleotide decoy comprises a sense strand, an antisense strand, a first carbon-containing linker that binds the 3′ end of the sense strand to the 5′ end of the antisense strand, and a second carbon-containing linker that binds the 5′ end of the sense strand to the 3′ end of the antisense strand, wherein the sense strand comprises the sequence 5′-CATTTCCCGTAAATC-3′ (SEQ ID NO: 1).

9. The method of claim 8, wherein the sense strand is up to 18 nucleotides long.

10. The method of claim 8, wherein the sense strand consists of the sequence 5′-CATTTCCCGTAAATC-3′ (SEQ ID NO: 1).

11. The method of any one of claims 8-10, wherein the antisense strand is at least partially complementary to the sense strand.

12. The method of any one of claims 8-10, wherein the antisense strand is fully complementary to the sense strand.

13. The method of any one of claims 8-12, wherein the sense strand has a phosphodiester backbone.

14. The method of any one of claims 8-13, wherein the antisense strand has a phosphodiester backbone.

15. The method of any one of claims 8-14, wherein the first carbon-containing linker comprises one or more ethylene glycolyl.

16. The method of any one of claims 8-14, wherein the first carbon-containing linker comprises hexaethylene glycolyl.

17. The method of any one of claims 8-16, wherein the second carbon-containing linker comprises one or more ethylene glycolyl.

18. The method of any one of claims 8-16, wherein the second carbon-containing linker comprises hexaethylene glycolyl.

19. The method of any one of claims 1-18, wherein the cancer is a head and neck cancer, breast cancer, ovarian cancer, lung cancer, pancreatic cancer, prostate cancer, skin cancer, cervical cancer, oral cancer, esophageal cancer, bladder cancer, leukemia, lymphoma, or a glioma.

20. The method of any one of claims 1-18, wherein the cancer is a head and neck cancer.

21. The method of any one of claims 1-18, wherein the cancer is lung cancer.

22. The method of any one of claims 1-18, wherein the cancer is colorectal cancer, pancreatic cancer, ovarian cancer, or melanoma.

23. The method of any one of claims 1-22. wherein the cancer is a squamous cell carcinoma.

24. The method of any one of claims 1-23, wherein the cancer is recurrent.

25. The method of any one of claims 1-24, wherein the cancer is metastatic.

26. The method of any one of claims 1-25, wherein the patient is a human.

27. The method of any one of claims 1-26, wherein the STAT3 double-stranded, cyclic oligonucleotide decoy is administered simultaneously with the immune checkpoint inhibitor.

28. The method of any one of claims 1-26, wherein the STAT3 double-stranded, cyclic oligonucleotide decoy and immune checkpoint inhibitor are administered sequentially.
